SEQUENCE LISTING (6 sequences)

SEQ ID NO: 1 - DNA, 23 bp, Artificial
  Synthetic DNA
ttgttttcaacaatgatgactgg

SEQ ID NO: 2 - DNA, 19 bp, Artificial
  Synthetic DNA
gcctgtgcagttgccatta

SEQ ID NO: 3 - DNA, 19 bp, Artificial
  Synthetic DNA
agccacatcgctcagacac

SEQ ID NO: 4 - DNA, 19 bp, Artificial
  Synthetic DNA
gcccaatacgaccaaatcc

SEQ ID NO: 5 - DNA, 18 bp, Artificial
  Synthetic DNA
gccttcaagagccacgtc

SEQ ID NO: 6 - DNA, 18 bp, Artificial
  Synthetic DNA
ccacgaactgtcgcatgt

(19) United States
(12) Patent Application Publication
(10) Pub. No.: US 2017/0349879 A1
(43) Pub. Date: Dec. 7, 2017

(54) METHOD FOR CULTURING NORMAL CELLS AND ODONTOMA CELLS CONTAINED IN ORAL TISSUE

(71) Applicant: CHIBA UNIVERSITY, Chiba (JP)

(72) Inventors: Hideki TANZAWA, Chiba (JP); Katsuhiro UZAWA, Chiba (JP); Atsushi KASAMATSU, Chiba (JP)

(21) Appl. No.: 15/539,107

(22) PCT Filed: Jul. 22, 2015
(86) PCT No.: PCT/JP2015/071425

§ 371 Date: Jun. 22, 2017

(30) Foreign Application Priority Data
Dec. 26, 2014 (JP) PCT/JP2014/084768

(51) Int. Cl. C12N 5/071; A61K 35/24; C12Q 1/68

(52) U.S. Cl. CPC C12N 5/0633; C12N 5/0632; C12Q 1/6806; A61K 35/24; C12N 2501/70; C12N 2533/90; C12Q 1/6853; C12N 2502/097

(57) ABSTRACT

The present invention provides a method for culturing odontoma cells or normal cells, wherein these cells are cultured in the presence of a Rho kinase inhibitor.

### TECHNICAL FIELD

The present invention relates to a method for culturing normal cells in oral tissue, particularly salivary gland cells, and a method for culturing odontoma cells.

### BACKGROUND ART

Saliva not only plays an important role in mucosal immunity in the oral cavity and esophagus, but is also deeply involved in ingestion, swallowing and other functions. For this reason, a reduction in the amount of salivary secretion caused by salivary gland atrophy due to aging, autoimmune diseases, radiation therapy and so on is responsible for causing various disorders, and the amount of salivary secretion is regarded as one of the important factors affecting the QOL of patients.

Many studies have now been made of regenerative medicine for salivary glands, although they are a far cry from practical application under the present circumstances. This is because it is very difficult to achieve primary culture of salivary gland cells, by way of example. Further, the salivary gland cell line (HSG) used in laboratories around the world was contaminated with HeLa cells (uterine cervical cancer cells), and this fact also serves as a factor responsible for slowing down studies.

Likewise, in the case of odontoma cells which are benign tumor cells, it is also difficult to achieve their initial culture and long-term culture, so that no long-term experiment can be performed on odontoma cells, and their developmental process has not yet been elucidated.

Namely, difficulties in primary culture and long-term culture of normal cells or benign tumor cells interfere with long-term experiments and therefore slow down studies.

On the other hand, it has been technically known that a Rock inhibitor is used to maintain the telomere length, thereby allowing long-term culture of primary cultured dermal cells (Non-patent Document 1: Chapman S. et al., 2010).

However, in the above document, success is limited only to primary cultured dermal cells, and there is no mention of oral tissue cells. This would be because it is difficult to achieve their primary culture due to the presence of many bacteria in the oral cavity.

Moreover, it has been known that an attempt to establish conditionally reprogrammed cells succeeded in malignant tumor cells by using a Rock inhibitor (Non-patent Document 2: Seema Agarwal, et al., Cancer Res, Apr. 15, 2013, 73, 1569).

However, Non-patent Document 2 is directed to malignant tumor cells with high autonomous growth ability, and there is no mention of any means required to achieve primary culture of normal cells and thereby allow their long-term culture.

### PRIOR ART DOCUMENTS

### Non-Patent Documents

Non-patent Document 1: Chapman S, Liu X, et al. Human keratinocytes are efficiently immortalized by a Rho kinase inhibitor. J Clin Invest. 120(7):2619-26, 2010.
Non-patent Document 2: Seema Agarwal, et al., Cancer Res, Apr. 15, 2013, 73, 1569

### SUMMARY OF THE INVENTION

### Problem to be Solved by the Invention

The object of the present invention is to provide a method for cell culture, which enables the long-term culture of normal cells in oral tissue and odontoma cells and also enables the construction of an experimental system leading to the elucidation of the mechanism for disease development.

### Means to Solve the Problem

As a result of extensive and intensive efforts made to solve the problems stated above, the inventors of the present invention have found that when a Rock inhibitor is used for culture of normal cells and odontoma cells, these cells can be cultured while maintaining their properties. This finding led to the completion of the present invention.

Namely, the present invention is as follows.

(1) A method for culturing normal cells or odontoma cells, wherein these cells are cultured in the presence of a Rho kinase inhibitor.
(2) The method according to (1) above, wherein the Rho kinase inhibitor is (R)-(+)-trans-N-(4-pyridyl)-4-(1-aminoethyl)-cyclohexanecarboxamide or a salt thereof.
(3) The method according to (1) or (2) above, wherein the normal cells are selected from the group consisting of salivary gland cells, oral mucosal epithelial cells, fibroblasts, vascular endothelial cells and cancer-associated fibroblasts.
(4) The method according to (1) or (2) above, wherein the normal cells are salivary gland cells.
(5) The method according to (3) or (4) above, wherein the salivary gland cells are those taken in vivo or those differentiated from stem cells.
(6) The method according to (1) or (2) above, wherein the odontoma cells are those taken in vivo or those differentiated from stem cells.
(7) A method for preparing salivary gland cells for use in transplantation, wherein the salivary gland cells are cultured in the presence of a Rho kinase inhibitor.
(8) The method according to (7) above, wherein the Rho kinase inhibitor is (R)-(+)-trans-N-(4-pyridyl)-4-(1-aminoethyl)-cyclohexanecarboxamide or a salt thereof.
(9) A material for regenerative medicine, which comprises salivary gland cells obtained by the method according to (7) or (8) above.

### Effects of the Invention

The present invention provides a method for culturing normal cells or odontoma cells. In the method of the present invention, a Rho kinase inhibitor can be used to establish primary culture of normal cells, particularly oral tissue-derived normal cells, and primary culture of odontoma cells, and further allows long-term stable culture of these cells while maintaining their morphology. Thus, the method of the present invention can be used for various cell-based experiments and is therefore very useful.

### BRIEF DESCRIPTION OF THE DRAWINGS

FIG. 1 shows the results obtained when salivary gland cells were cultured using a Rock inhibitor.

FIG. 2 shows the results analyzed for the expression level of amylase in salivary gland cells.

FIG. 3 shows the results of protein expression analysis of amylase in salivary gland cells.

FIG. 4 shows the results analyzed for morphological changes in salivary gland cells.

FIG. 5 shows the results of karyotype analysis on the chromosomes of salivary gland cells.

FIG. 6 shows the results analyzed for cell proliferation, cell death and immortalization in salivary gland cells.

FIG. 7 shows the results of hematological tests in mice.

FIG. 8 shows the time course of body weight changes in mice administered intraperitoneally with a Rock inhibitor.

FIG. 9 shows the results of histopathological tests performed on the organs of mice administered intraperitoneally with a Rock inhibitor.

FIG. 10 shows the amount of salivary secretion after transplantation of salivary gland cells in radiation-induced salivary gland atrophy model rats.

FIG. 11 shows the results obtained when odontoma cells were cultured.

FIG. 12 shows the results obtained when oral mucosal epithelial cells were cultured.

FIG. 13 shows the results obtained when fibroblasts were cultured.

FIG. 14 shows the results obtained when cancer-associated fibroblasts were cultured.

FIG. 15 shows the results obtained when vascular endothelial cells were cultured.

### DESCRIPTION OF EMBODIMENTS

The present invention is directed to a method for culturing odontoma cells or normal cells, characterized in that these cells are cultured in the presence of a Rho kinase inhibitor. As a result of using a Rho kinase inhibitor for culture of odontoma cells or culturing normal cells (e.g., salivary gland cells, oral mucosal epithelial cells, fibroblasts, vascular endothelial cells, cancer-associated fibroblasts (CAFs)) in the presence of a Rock inhibitor, the inventors of the present invention not only have established a long-term culture method allowing subculture of odontoma cells or normal cells, which are difficult to culture for primary culture, while maintaining the characteristics of odontoma or the characteristics of normal cells, but also have succeeded in primary culture of normal cells, which are difficult to culture for long-term culture, thereby completing the present invention.

When cultured in the absence of a Rho kinase inhibitor, odontoma cells would change their cell morphology and therefore die after 2 or 3 passages of subculture. In contrast, addition of a Rho kinase inhibitor allowed their culture while maintaining their morphology. Likewise, in the case of normal cells (e.g., salivary gland cells, oral mucosal epithelial cells, fibroblasts, cancer-associated fibroblasts, vascular endothelial cells) which are difficult to culture for primary culture, their primary culture was also able to succeed when using a Rho kinase inhibitor.

### 1. Cells

Odontoma cells, which are one of the cells used in the present invention, are a kind of epithelial tumor associated with induction of ectodermal mesenchymal tissue, and can be obtained by being taken as a portion of the excised matter during odontoma excision.

The term “normal cells” is intended to mean cells which are not malignant tumor cells. In particular, the present invention provides a culture method useful for cells which are difficult to culture for primary culture and long-term culture. Such cells which are difficult to culture for primary culture and long-term culture may be exemplified by normal cells in oral tissue. Oral tissue refers to a tissue constituting the oral cavity and containing nerves and blood vessels therein, as exemplified by bucca, lip, palate, salivary glands, gum and so on. Examples of normal cells in oral tissue include salivary gland cells, oral mucosal epithelial cells, fibroblasts, cancer-associated fibroblasts (CAFs), vascular endothelial cells and so on.

In addition, non-oral cells which are difficult to culture for primary culture and long-term culture may be exemplified by prostate cells, cancer-associated fibroblasts (CAFs), vascular endothelial cells and so on.

Such normal cells can be obtained from normal tissue taken in vivo through crushing, trypsin treatment, etc.

Among oral tissues, salivary gland cells when derived from biomaterials may be taken from human labial glands. After being taken, the labial glands are washed with culture medium or physiological saline, etc., and then cut into small pieces for use as a source material for culture.

Likewise, oral mucosal epithelial cells may also be obtained by culturing epithelial cells taken from outpatients during wisdom tooth extraction. Fibroblasts, cancer-associated fibroblasts and vascular endothelial cells may be taken for culture from normal tissue and malignant tumor tissue, respectively, during surgery of malignant tumor in oral tissue.

For surgical removal of malignant tumor, the tumor is generally removed as a mass with a margin area including normal tissue. Thus, fibroblasts may be separated and cultured from normal tissue in the removed mass, while cancer-associated fibroblasts and vascular endothelial cells may be separated from malignant tumor tissue in the removed mass. Although cancer-associated fibroblasts and vascular endothelial cells are taken from malignant tumor tissue, they are normal cells and are separated alone for culture.

The cells thus obtained are then seeded and cultured in culture vessels for primary culture.

The cells to be used in the present invention may be subcultured before use. The number of passages during subculture is not limited in any way, but it is preferably 1 to 10, more preferably 2 to 7, and even more preferably 2 to 5. As cells having been subcultured, commercially available established normal cells may also be used in the method of the present invention.

### 2. Rho Kinase Inhibitors

Rho kinase (ROCK: Rho-associated coiled-coil containing protein kinase) is a kind of protein kinase and an enzyme involved in the regulatory mechanism for cellular responses based on Rho-ROCK signaling. The Rho kinase inhibitor to be used in the present invention is not limited in any way and may be selected freely, as long as it is a substance inhibiting such Rho kinase. In the present invention, examples of a Rho kinase inhibitor (hereinafter also referred to as a “Rock inhibitor”) include Y-27632, HA1077, HA1100, Y-39983 and so on.

Y-27632 is a hydrochloride salt of (R)-(+)-trans-N-(4-pyridyl)-4-(1-aminoethyl)-cyclohexanecarboxamide (represented by formula I shown below) and is known as a substance which inhibits ROCK signaling system-mediated contraction of vascular smooth muscle, cancer cell invasion and cell differentiation control.

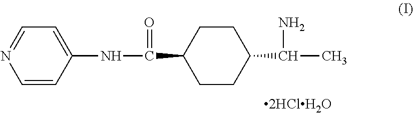 [SMILES: CC(N)[C@H]1CC[C@H](C(=O)Nc2ccncc2)CC1]

Y-27632 is commercially available (Wako Pure Chemical Industries, Ltd., Japan) and can be readily obtained.

HA1077 is a hydrochloride salt of 1-(5-isoquinolinesulfonyl)homopiperazine (represented by formula II shown below).

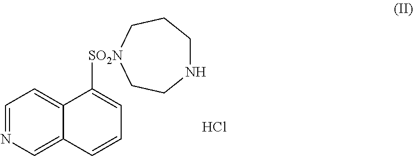

HA-1100 is commercially available (ALEXIS BIOCHEMICALS) and can be readily obtained (represented by formula III shown below).

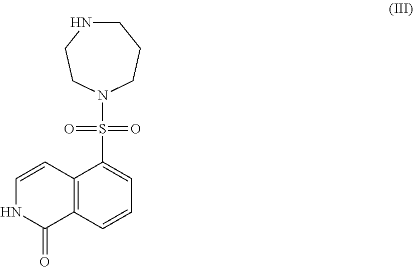 [SMILES: O=c1[nH]ccc2c(S(=O)(=O)N3CCCNCC3)cccc12]

Y-39983 is commercially available (Medchem Express) and can be readily obtained (represented by formula IV shown below).

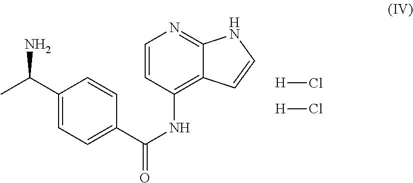

### 3. Culture

The above odontoma cells or normal cells are cultured in the presence of a Rock inhibitor. The expression “cultured in the presence of a Rock inhibitor” is intended to mean being cultured in a state where a Rock inhibitor and cells to be cultured can be contacted with each other, and includes all embodiments where a Rock inhibitor is added to a medium containing odontoma cells or normal cells, where odontoma cells or normal cells and a Rock inhibitor are added together to a medium, and where odontoma cells or normal cells are seeded into a medium containing a Rock inhibitor.

The liquid medium for cell culture to be used for culture of odontoma cells or normal cells is not limited in any way, and examples include known basal media for cell culture, such as Dulbecco's modified Eagle medium (DMEM), Williams' E medium, Ham's F-10 medium and F-12 medium, RPMI-1640 medium, 199 medium, Keratinocyte-SFM medium, HepatoZYME-SFM medium and so on, which may optionally be supplemented with an additive(s) appropriate for culture of the above cells.

Examples of such an additive include growth factors or cell growth factors, antibiotics, organic compounds, fetal bovine serum and so on. Examples of growth factors or cell growth factors available for use include fibroblast growth factor (FGF), transforming growth factor-β (TGF-β), insulin-like growth factor (IGF), vascular endothelial growth factor (VEGF), etc.

In addition, odontoma cells or normal cells may be cultured in a state being covered with, e.g., a collagen gel or an agarose gel, each containing a liquid medium for cell culture. This allows protection of the cells from physical damage applied to the cells during medium replacement, etc.

The temperature during culture may be set to a temperature normally applied during culture of animal cells, for example, may be to set to 36° C. to 37° C. Culture may be conducted in an incubator under an atmosphere of CO₂ at a concentration of 5% to 10%, preferably at a concentration of 5%.

Confirmation of whether the cells thus obtained are odontoma cells or normal cells may be accomplished, for example, by using cell surface markers, intracellular markers (e.g., mRNAs, proteins or enzymes present within the desired cells) and/or extracellular markers (e.g., peptides, proteins, enzymes or compounds secreted into the extracellular environment) as indicators. For marker detection, any technique may be used and examples include labeled antibody-based techniques (e.g., staining, flow cytometry, ELISA), enzyme activity-based staining techniques, RT-PCR techniques, etc.

The normal cells obtained as above can be used in regenerative medicine as cells for medical biomaterials.

### 4. Method for Regenerative Medicine or Treatment

As another embodiment, the present invention is further directed to a method for treatment and/or regenerative medicine, characterized in that normal cells prepared by the method of the present invention are transplanted into a patient. The site of transplantation and the route of administration may be varied as appropriate depending on the type of cells. The number of cells to be transplanted is 1.0×10⁵ to 1.0×10⁸ cells, and preferably 1.0×10⁷ to 1.0×10⁸ cells.

For example, when intended for regenerative medicine of salivary glands, the above method can facilitate the regeneration of salivary glands in a patient who has developed salivary gland atrophy due to aging, autoimmune diseases, radiation therapy and so on. The above transplantation may be accomplished, for example, by seeding the above cells in a collagen gel or the like and locating the gel in the salivary glands of a patient with salivary gland hypofunction.

The present invention enables the long-term culture of odontoma cells or normal cells (e.g., salivary gland cells, oral mucosal epithelial cells, fibroblasts, vascular endothelial cells, cancer-associated fibroblasts (CAFs)). For this reason, the present invention enables the construction of an experimental system leading to the elucidation of the mechanism for disease development, and will be able to contribute to advances in future studies.

In terms of the fact that odontoma cells are difficult to culture, very few experiments have been conducted on a disease called odontoma. The inventors of the present invention have succeeded in long-term stable culture of odontoma cells by using a Rho kinase inhibitor during their primary culture. For this reason, once this culture method has been applied to pursue further studies, it will be able to contribute to future studies.

### EXAMPLES

The present invention will be further described in more detail by way of the following illustrative examples, although the present invention is not limited to these examples.

### Example 1

Establishment of a method for primary culture of salivary glands

### <Material and Method>

### 1. Culture of Salivary Gland Cells Using a Rock Inhibitor (Y27632, Wako, Code No. 251-00514)

### 1.1 Method for Primary Culture of Salivary Gland Cells

(i) Patient-derived salivary gland cells for use as a source material were taken in a required amount from the labial glands of each patient.

(ii) The cells were washed three times with phosphate-buffered physiological saline (PBS) (supplemented with 10 units/ml of penicillin and 100 μg/ml of streptomycin: Sigma-Aldrich).

(iii) The cells were cut into small pieces with sterilized iris scissors and a surgical knife (No. 11).

(iv) After addition of trypsin (TrypLE Select Enzyme, Gibco, Code No. A12177-01), the cells were shaken at 37° C. for 30 minutes.

(v) The cells were mixed with Dulbecco's modified Eagle medium (DMEM: Sigma-Aldrich) (supplemented with 10% fetal bovine serum: Nichirei Bioscience Inc., Japan, 10 units/ml of penicillin and 100 μg/ml of streptomycin), and centrifugation (at 1000 rpm for 5 minutes) was repeated three times.

(vi) The cells were mixed with DMEM (supplemented with 10% fetal bovine serum, 10 units/ml of penicillin, 100 μg/ml of streptomycin, 10 μM of the Rock kinase inhibitor and 0.3 μM of L-ascorbic acid 2-phosphate: Sigma-Aldrich) (DMEM+) and then seeded into collagen type 1-coated cell culture dishes (IWAKI).

(vii) The cells were washed with PBS once every three days to replace the above medium.

### 1.2 Method for Subculture of Cultured Cells

(i) The cultured cells were subcultured at 80% confluency. The cells were washed three times with phosphate-buffered physiological saline.

(ii) After trypsin was added to the culture vessels, the cells were cultured for 7 minutes at 37° C.

(iii) The cells were mixed with DMEM+ and centrifuged (at 1000 rpm for 5 minutes).

(iv) After removal of the supernatant, the cells were diluted with DMEM+ and seeded into collagen type 1-coated cell culture dishes.

### 2. Analysis of Amylase Expression Levels

### 2.1 Expression Analysis of Amylase

Salivary gland cells were cultured in the presence or absence of a Rock inhibitor and provided for quantitative RT-PCR assay with a LightCycler 480 (Roche) to analyze amylase expression in these salivary gland cells. The primers used for amylase are as shown below, and the universal probe used was #18.

AMF01 (forward):
(SEQ ID NO: 1)
5′-TTGTTTTCAACAATGATGACTTG-3′
AMR01 (reverse):
(SEQ ID NO: 2)
5′-GCCTGTGCAGTTGCCATTA-3′

The primers used for glyceraldehyde-3-phosphate dehydrogenase (GAPDH) serving as an endogenous control are as shown below, and the universal probe used was #60.

GAPDHF01 (forward):
(SEQ ID NO: 3)
5′-AGCCACATCGCTCAGACAC-3′
GAPDHR01 (reverse):
(SEQ ID NO: 4)
5′-GCCCAATACGACCAAATCC-3′

The PCR reaction was conducted in accordance with standard protocols by using a mixture (final reaction volume: 20 μl) containing 10 μl of Probe master (Roche), 0.2 μl of each probe master and the primers. The reaction conditions were set to initial denaturation at 95° C. for 5 minutes and 50 amplification cycles of denaturation at 95° C. for 10 seconds, annealing at 60° C. for 30 seconds and elongation reaction at 60° C. for 60 seconds. The mRNA level of the desired gene was expressed as a relative value to the expression level of GAPDH used as a control.

### 2.2 Protein Expression Analysis of Amylase by Immunocytological Fluorescent Staining

(i) Salivary gland cells cultured on glass slides were fixed by being treated for 10 minutes with 4% paraformaldehyde (WaKo).

(ii) The fixed cells were washed three times with Tris-buffered saline (TBS: Sigma-Aldrich).

(iii) The fixed cells were permeated with 0.5% Triton X-100 (WaKo) for 15 minutes.

(iv) The fixed cells were reacted for 30 minutes with TBS containing 1% bovine serum albumin (Sigma-Aldrich).

(v) The fixed cells were washed three times with TBS.

(vi) A primary antibody (anti-goat amylase (Santa Cruz)) was mixed with TBS and reacted at a concentration of 1:200 at 4° C. for 16 hours.

(vii) The cells were washed three times with TBS.

(viii) Anti-goat Alexa Floor 647 (Millipore) was mixed with phosphate-buffered physiological saline and reacted at a concentration of 1:200 at room temperature for 1 hour.

(ix) The cells were washed three times with TBS.

(x) The cells were mounted with DAPI-containing mounting medium (DaKo).

(xi) The cells were observed under a confocal microscope (FV10i-LIV: OLYMPUS).

### 3. Analysis of Cytomorphological Changes

In this section, cytomorphological changes caused by the presence or absence of a Rock inhibitor were analyzed by immunocytological fluorescent staining of the cytoskeleton.

### 3.1 Analyte Treatment

Analytes were treated by the same experimental procedures as shown in (2.2) above. As an antibody, Acti-stain 670 fluorescent Phallodin (F-actin) (Cytoskeleton) was reacted at room temperature for 1 hour.

### 4. Confirmation of Cytotoxicity (Safety) in Consideration of Clinical Application

### 4.1 Karyotype Analysis

Primary cultured salivary gland cells were cultured in a medium containing a Rock inhibitor and analyzed for the presence or absence of their genetic defect(s) by karyotype analysis. The karyotype analysis test was conducted by Q band technique.

### 4.2 Analysis of Salivary Gland Cells

Primary cultured salivary gland cells were cultured in a medium containing a Rock inhibitor and analyzed for their proliferation by Western blotting of proliferating cell nuclear antigen (PCNA: Santa Cruz) and for their apoptosis by Western blotting of caspase-3 (CST). For analysis of their immortalization, the expression of telomerase reverse transcriptase (TERT) was analyzed by quantitative RT-PCR assay.

Quantitative RT-PCR of TERT was conducted in the same manner as shown in (2.1) above. The primers used are as shown below.

TERTF01: (forward):
(SEQ ID NO: 5)
5′-GCCTTCAAGAGCCACGTC-3′
TERTR01: (reverse):
(SEQ ID NO: 6)
5′-CCACGAACTGTCGCATGT-3′

The universal probe used was #19.

The protein levels of PCNA and caspase-3 were measured by Western blotting. In addition, α-tubulin was used as an endogenous control.

(i) A protein extract was electrophoresed on a 4% to 12% polyacrylamide gel (Invitrogen).

(ii) The proteins were transferred onto a nitrocellulose membrane (Invitrogen).

(iii) The membrane was washed with Tris-buffered saline containing 0.1% Tween-20 (WaKo) (TBST), and then blocked with Blocking One (Nacalai Tesque, Inc., Japan) for 30 minutes.

(iv) The membrane was reacted at 4° C. for 16 hours with PCNA antibody (Santa Cruz), caspase-3 antibody (Cell Signaling Technology) or α-tubulin antibody (Santa Cruz).

(v) The membrane was washed three times with TBST.

(vi) The membrane was reacted at room temperature for 1 hour with horseradish peroxidase (HRP)-conjugated secondary antibody (Promega) and then treated with Super Signal West Pico Chemiluminescent substrate (Thermo) to develop signals for visualization purposes. The intensity of signals was quantified with a CS analyzer (ATTO) and expressed as a relative value in comparison with the protein level of α-tubulin measured as a control.

4.3 Histopathological Analysis of Organ Toxicity in Mice Administered Intraperitoneally with a Rock Inhibitor

Animal used: B6C3F1/Crlj mice (CLEA Japan, Inc., Japan)

n=12 for each group (male: 6, female: 6), n=4 for the control group (male: 2, female: 2)

Drug: Y-27632

Dose: 10 mg/kg/day (0.25 mg/day for 25 g body weight)

Y-27632 was dissolved in PBS and intraperitoneally administered. The control group was administered with an equal volume of PBS.

Administration period: administered once a day for successive 14 days.

Half of the mice in each group were monitored for follow-up purposes for 14 days after administration.

### Groups to be Measured:

(i) Groups receiving a Rock inhibitor (RI+): n=12 (male: 6, female: 6)

A. Group subjected to blood collection and autopsy at the time of the completion of administration (at 14 days after the initiation of administration). n=6 (male: 3, female: 3)

B. Group subjected to blood collection and autopsy at the time of the completion of administration (at 28 days after the initiation of administration). n=6 (male: 3, female: 3)

(ii) Groups receiving no Rock inhibitor (RI−): n=12 (male: 6, female: 6)

A. Group subjected to blood collection and autopsy at the time of the completion of administration (at 14 days after the initiation of administration). n=6 (male: 3, female: 3)

B. Group subjected to blood collection and autopsy at the time of the completion of administration (at 28 days after the initiation of administration). n=6 (male: 3, female: 3)

(iii) Control group: n=4 (male: 2, female: 2)

*: Group for use in the preparation of base data, which received no treatment (e.g., drug administration) and was subjected to blood collection and autopsy on the same day as the day of initiation of administration in the other groups.

### Items to be Measured:

### 1) General Condition

The mice were visually observed for their general condition once a day.

### 2) Measurement of Body Weight and Feed/Water Intake

The mice were measured daily for their body weight and feed/water intake.

### 3) Hematological Tests

Hematological tests were performed on each mouse before administration and after administration (on the 14th day). * In each group, 3 male and 3 female mice were subjected to blood collection and autopsy on the 14th day of administration. The remaining 3 male and 3 female mice were withdrawn from administration over 14 days after the completion of administration, during which they were observed for their general condition and measured for their body weight and feed/water intake. These mice were subjected to blood examination and autopsy on the 28th day after the initiation of administration.

Blood counting: red blood cell count, white blood cell count, platelet count, hematocrit value, red blood cell fraction.
Biochemical parameters: TP, ALB, CRE, Na, K, GOT, GPT, T-CHO, Glu

### Autopsy

On the 14th day after the completion of administration, 3 male and 3 female mice from each group were subjected to autopsy. The remaining 3 male and 3 female mice were subjected to autopsy on the 28th day after being monitored for follow-up purposes.

### Visual Observation

Pathological observation: For visible lesions and heart, lung, pancreas, liver, spleen, kidney, genital gland and skeletal muscle, pathological observation was conducted by HE staining.

### Results

### 1. Culture of Salivary Gland Cells Using a Rock Inhibitor

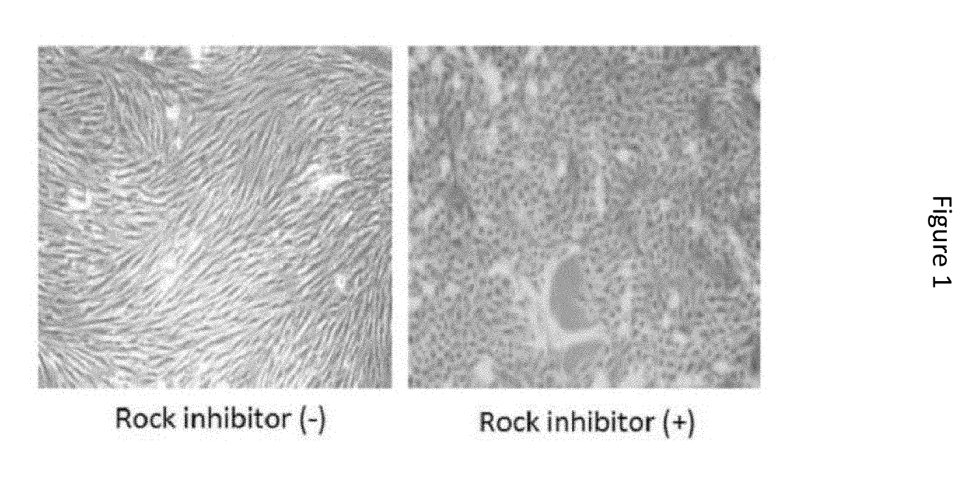

When salivary gland cells were cultured in a standard medium, they showed fibroblast-like changes in cell morphology every time their subculture was repeated (FIG. 1, Rock inhibitor (−)) and also showed an extreme reduction in their proliferation. Upon culture in a Rock inhibitor-containing medium, salivary gland cells were found to maintain their cell morphology arranged in a cobblestone pattern even when repeatedly subcultured (FIG. 1, Rock inhibitor (+)).

2. Salivary Gland Cells were Cultured in the Presence or Absence of a Rock Inhibitor to Analyze the Expression Level of Amylase in the Salivary Gland Cells.

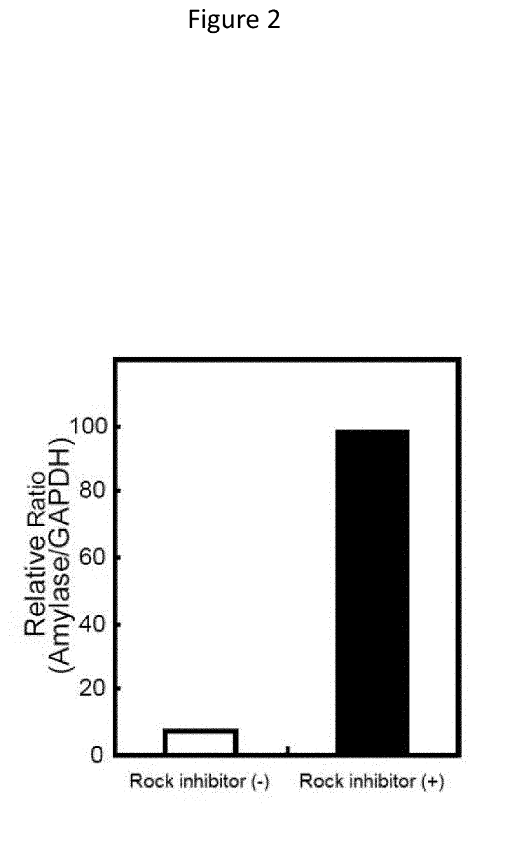

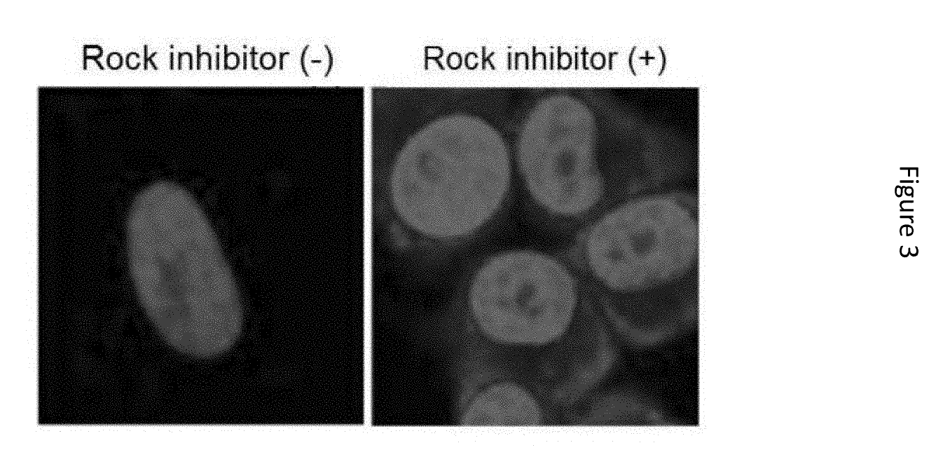

Even when salivary gland cells were cultured and also repeatedly subcultured in a Rock inhibitor-containing medium, they were found to show significantly high mRNA (FIG. 2) and protein (FIG. 3) expression of amylase, thus indicating that the expression of amylase was maintained at high level by the action of the Rock inhibitor.

3. Cytomorphological Changes Caused by the Presence or Absence of a Rock Inhibitor were Analyzed by Immunocytological Fluorescent Staining of the Cytoskeleton.

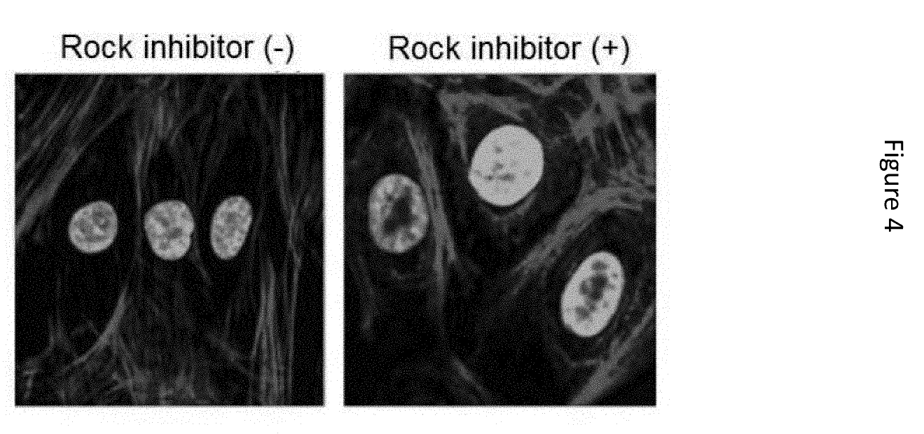

For verification of morphological changes, immunocytological fluorescent staining was conducted by means of the cytoskeleton F-actin. When cultured in a Rock inhibitor-free medium, salivary gland cells clearly showed a fibroblast-like skeleton. In contrast, salivary gland cells cultured in a Rock inhibitor-containing medium showed a honeycomb-like skeleton (FIG. 4).

### 4. Cytotoxicity (Safety) was Confirmed in Consideration of Clinical Application.

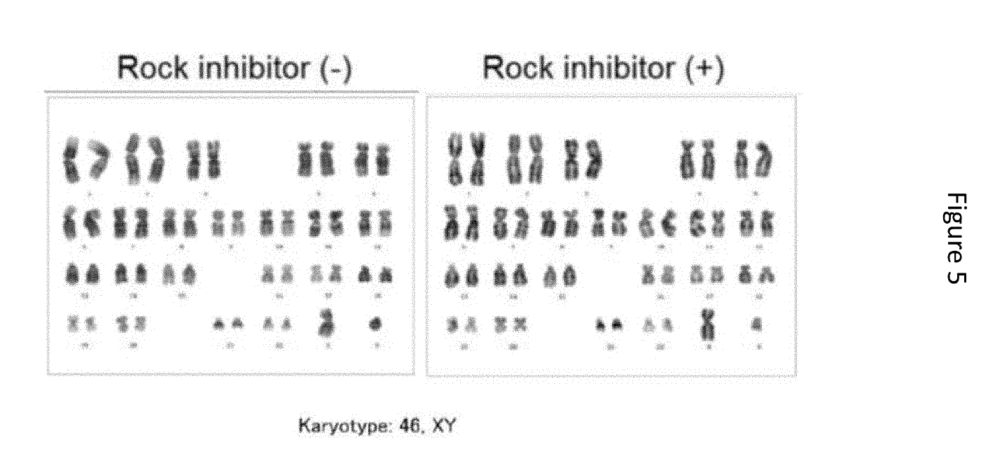

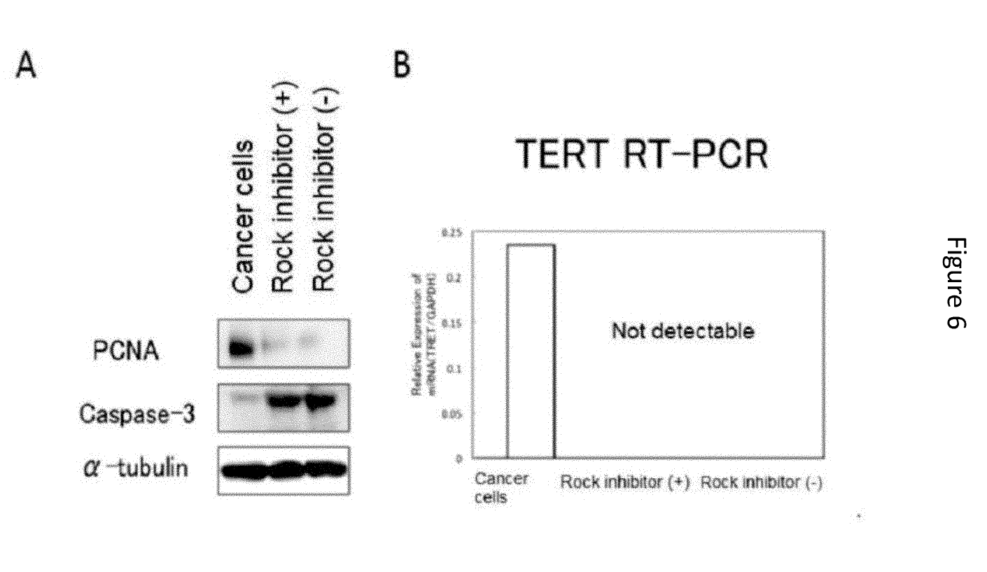

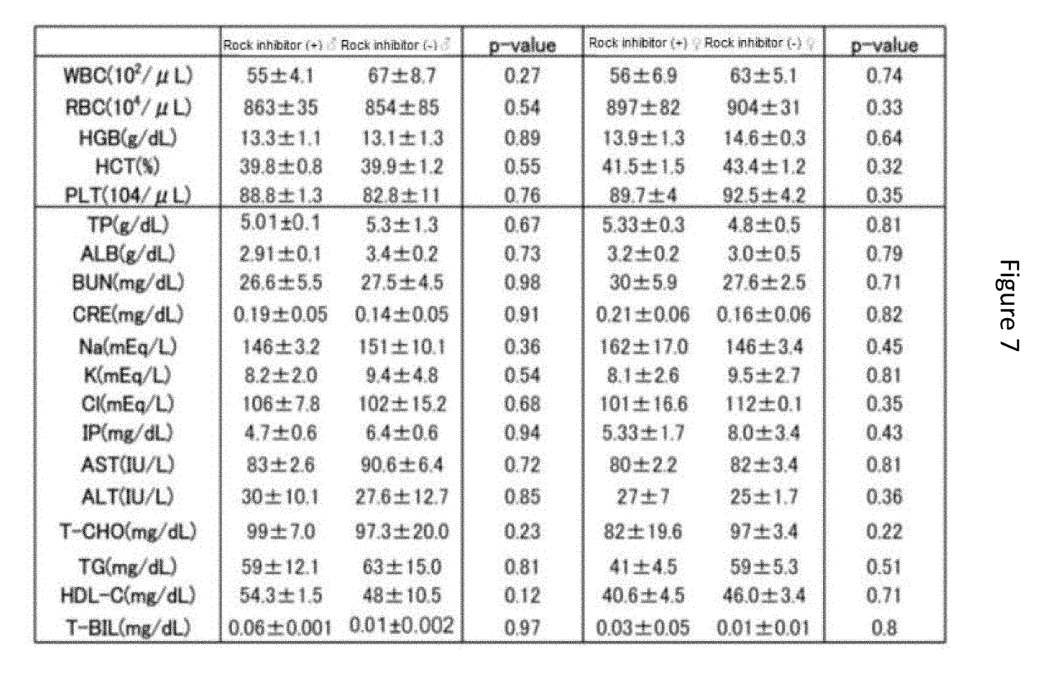

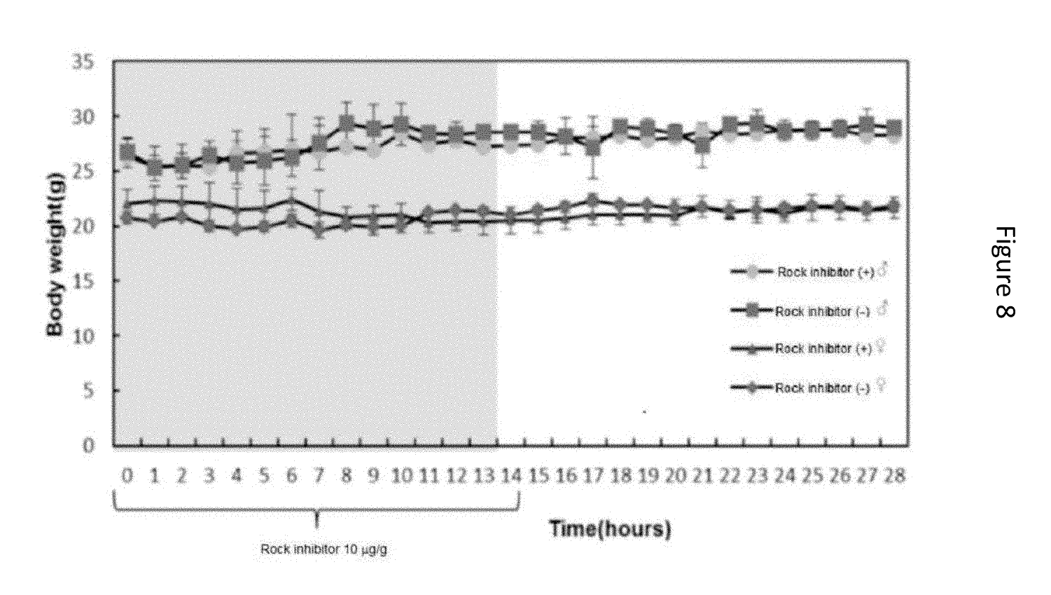

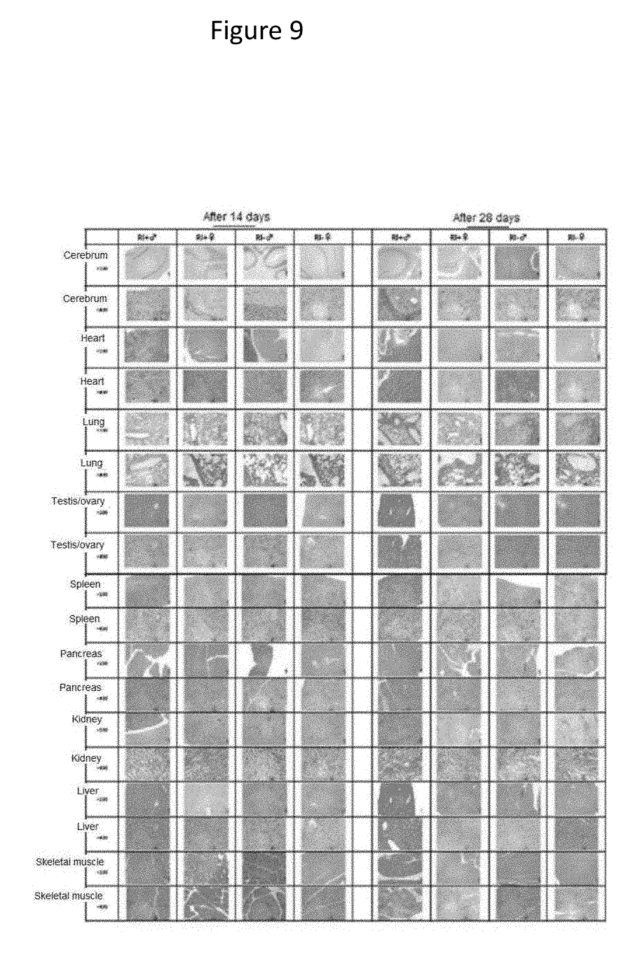

4.1. Primary cultured labial gland cells were examined by karyotype analysis for the presence or absence of their chromosomal defect(s) caused when cultured in a Rock agonist-containing medium. No genetic defect was observed in this karyotype analysis (FIG. 5).
4.2. Even when salivary gland cells were cultured in a Rock inhibitor-containing medium, no difference was observed in PCNA or caspase-3 expression (FIG. 6A). Likewise, no difference was also observed in TERT expression, and TERT expression was not detectable in salivary gland cells (FIG. 6B).
4.3. A Rock inhibitor was intraperitoneally administered to mice and examined for its toxicity to the mice by comparison of hematological tests (FIG. 7), changes in body weight (FIG. 8) and influences on individual mouse organs (FIG. 9). No abnormal findings were observed in the hematological tests, the time course of body weight changes and the histopathological examination, thus confirming that the Rock inhibitor was safe.

### Example 2

### Test for Transplantation and Regeneration of Salivary Gland Cells

Female nude rats at 4 weeks of age (Oriental Yeast Co., Ltd., Japan) (hereinafter referred to as nude rats) were irradiated once with 15 Gy radiation on their submandibular glands (SMGs) to prepare rats with radiation-induced hyposalivation.

### Treatment of Rats

Experimental control groups were divided into the following three groups: a group receiving atelocollagen alone (hereinafter referred to as the control group), a group receiving both atelocollagen and cells (hereinafter referred to as the cell group), and a non-irradiated group receiving no radiation (hereinafter referred to as the normal group).

Atelocollagen was prepared from an atelocollagen gel (IPC50 KOKEN) and treated with a medium to have a pH in the neutral range immediately before injection through the Wharton's duct. The control group and the cell group were injected with atelocollagen (200 μl) and with atelocollagen (200 μl) and about 2.0×10⁶ cells (GFP rat SMG cells), respectively, through their Wharton's ducts at both sides immediately after irradiation.

### Test for Measurement of Salivary Glands

These three groups, i.e., the control group, the cell group and the normal group were subjected to a salivary measurement test. For salivary measurement, these groups were compared for their salivary flow rate (hereinafter abbreviated as SFR). Pilocarpine nitrate (Lot No. 081M1532V SIGMA-ALDRICH) was used for salivary measurement. Pilocarpine nitrate was prepared at 1.0 mg/ml with physiological saline immediately before use and administered at a dose of 5 mg/kg to each nude rat via the intraperitoneal route. The flow rate of saliva was determined by measuring the amount of saliva for 30 minutes, and the comparison results are shown in FIG. 10.

As shown in FIG. 10, when compared to the normal group, the control group and the cell group were found to reduce their salivary flow rate due to the influence of irradiation. However, in terms of the total amount of SFR at 8 weeks after irradiation, the amount of saliva was increased in the cell group when compared to the control group, thus indicating that salivary glands were found to recover their function.

### Example 3

### <Material and Method>

### 1. Cell Culture Using a Rho Kinase Inhibitor (Y27632, Wako, Code No. 251-00514)

### (1) Cells

Odontoma cells, oral mucosal epithelial cells, fibroblasts, and cancer-associated fibroblasts (CAFs)

### (2) Method

(i) Patient-derived cells for use as a source material were taken in a required amount as described above.

(ii) The cells were washed three times with phosphate-buffered physiological saline (PBS) (supplemented with 10 units/ml of penicillin and 100 μg/ml of streptomycin: Sigma-Aldrich).

(iii) The cells were cut into small pieces with sterilized iris scissors and a surgical knife (No. 11).

(iv) After addition of trypsin (TrypLE Select Enzyme, Gibco, Code No. A12177-01), the cells were shaken at 37° C. for 30 minutes.

(v) The cells were mixed with Dulbecco's modified Eagle medium (DMEM: Sigma-Aldrich) (supplemented with 10% fetal bovine serum: Nichirei Bioscience Inc., Japan, 10 units/ml of penicillin and 100 μg/ml of streptomycin), and centrifugation (at 1000 rpm for 5 minutes) was repeated three times.

(vi) The cells were mixed with DMEM (supplemented with 10% fetal bovine serum, 10 units/ml of penicillin, 100 μg/ml of streptomycin, 10 μM of the Rock kinase inhibitor and 0.3 μM of L-ascorbic acid 2-phosphate: Sigma-Aldrich) (DMEM+) and then seeded into collagen type I-coated cell culture dishes (IWAKI).

(vii) The cells were washed with PBS once every three days to replace the above medium.

(viii) The cultured cells were subcultured at 80% confluency. The cells were washed three times with phosphate-buffered physiological saline.

(ix) After trypsin was added to the culture vessels, the cells were cultured for 7 minutes at 37° C.

(x) The cells were mixed with DMEM+ and centrifuged (at 1000 rpm for 5 minutes).

(xi) After removal of the supernatant, the cells were diluted with DMEM+ and seeded into collagen type I-coated cell culture dishes.

### 2. Cell Culture Using a Rho Kinase Inhibitor (Y27632, Wako, Code No. 251-00514)

### (1) Cells

Vascular Endothelial Cells

### (2) Method

(i) Patient-derived cells for use as a source material were taken in a required amount as described above.

(ii) The cells were washed three times with phosphate-buffered physiological saline (PBS) (supplemented with 10 units/ml of penicillin and 100 μg/ml of streptomycin: Sigma-Aldrich).

(iii) The cells were cut into small pieces with sterilized iris scissors and a surgical knife (No. 11).

(iv) The cells were added to collagenase (032-10534: Wako) and shaken at 37° C. for 60 minutes.

(v) The cells were diluted with an equal volume of fetal bovine serum (Nichirei Bioscience Inc., Japan) and centrifuged (at 1000 rpm for 10 minutes), and then centrifuged (at 1000 rpm for 5 minutes) three times with PBS, followed by addition of MACS buffer and centrifugation (at 1000 rpm for 5 minutes).

(vi) Magnetic beads (Dynabeads CD31 Endotherial cell, 11155D: Invitrogen) were used to separate the cells.

(vii) The cells were mixed with EGM-2MV (Lonza) (supplemented with 20% fetal bovine serum, 10 units/ml of penicillin, 100 μg/ml of streptomycin, and 10 μM of the Rock kinase inhibitor) and then seeded into fibronectin-coated cell culture dishes (IWAKI).

(viii) The cells were washed with PBS once every three days to replace the above medium.

(ix) The cultured cells were subcultured at 80% confluency. The cells were washed three times with phosphate-buffered physiological saline.

(x) After trypsin was added to the culture vessels, the cells were cultured for 7 minutes at 37° C.

(xi) The cells were mixed with EGM-2MV and centrifuged (at 1000 rpm for 5 minutes).

(xii) After removal of the supernatant, the cells were diluted with EGM-2MV and seeded into fibronectin-coated cell culture dishes.

### 3. Results

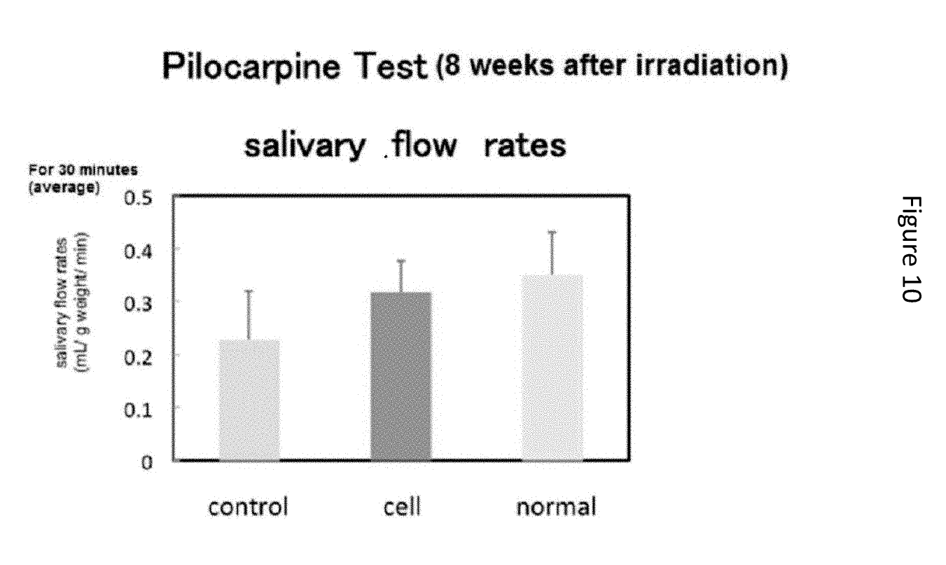

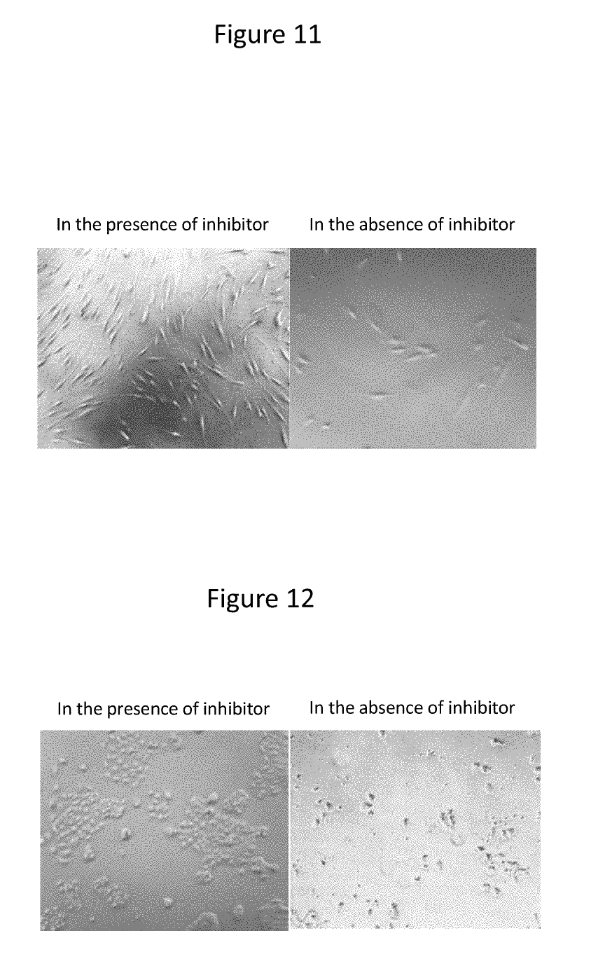

Cell morphology was observed for comparison with the control (in the absence of the inhibitor). As a result, upon culture in an inhibitor-containing medium, the cells were found to maintain their cell morphology even when repeatedly subcultured. Upon culture in an inhibitor-free medium, the cells were found to change their cell morphology after 2 or 3 passages of subculture, so that it was difficult to continue their culture. Moreover, some cells had already changed their cell morphology before subculture (FIGS. 11 to 15).

FIG. 11: odontoma cells, at passage 5 in the presence of the inhibitor and at passage 0 in the absence of the inhibitor

FIG. 12: oral mucosal epithelial cells, at passage 5 in the presence of the inhibitor and at passage 0 in the absence of the inhibitor

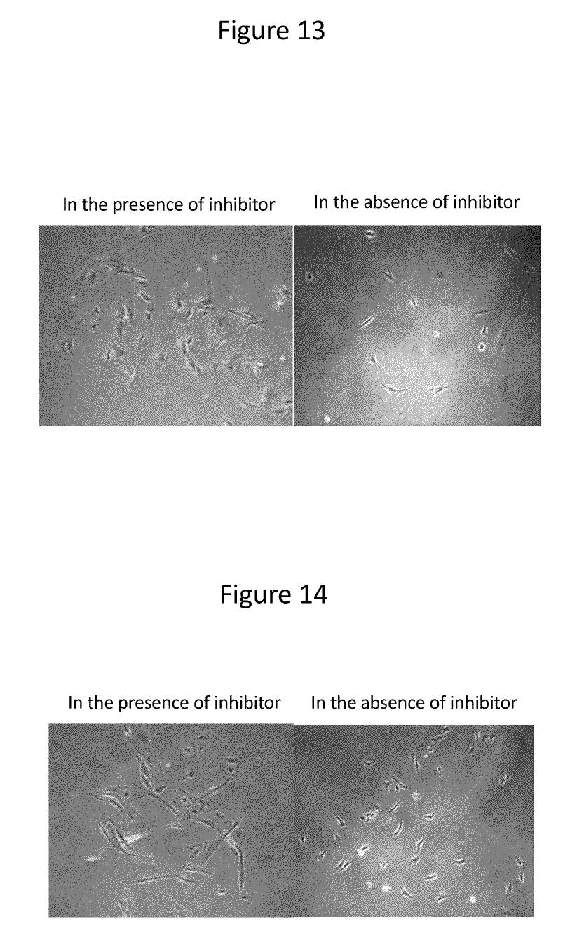

FIG. 13: fibroblasts, at passage 7 in the presence of the inhibitor and at passage 2 in the absence of the inhibitor

FIG. 14: cancer-associated fibroblasts, at passage 7 in the presence of the inhibitor and at passage 1 in the absence of the inhibitor

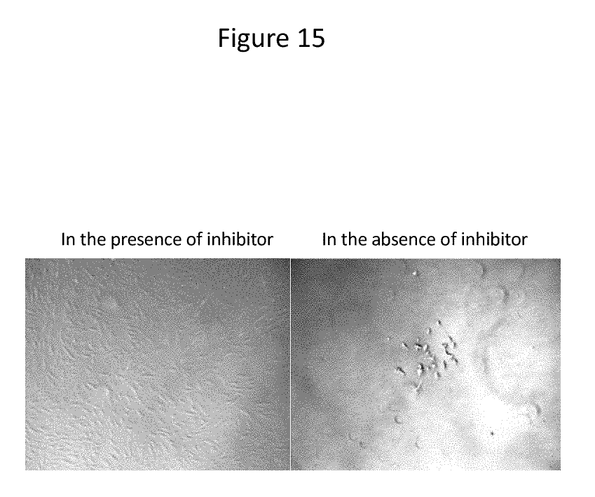

FIG. 15: vascular endothelial cells, at passage 8 in the presence of the inhibitor and at passage 0 in the absence of the inhibitor

### Sequence Listing Free Text

SEQ ID NO: 1: synthetic DNA
SEQ ID NO: 2: synthetic DNA
SEQ ID NO: 3: synthetic DNA
SEQ ID NO: 4: synthetic DNA
SEQ ID NO: 5: synthetic DNA
SEQ ID NO: 6: synthetic DNA

1. A method for culturing normal cells or odontoma cells, wherein a cell sample consisting of these cells is cultured in the presence of a Rho kinase inhibitor.

2. The method according to claim 1, wherein the Rho kinase inhibitor is (R)-(+)-trans-N-(4-pyridyl)-4-(1-aminoethyl)-cyclohexanecarboxamide or a salt thereof.

3. The method according to claim 1, wherein the normal cells are selected from the group consisting of salivary gland cells, oral mucosal epithelial cells, fibroblasts, vascular endothelial cells and cancer-associated fibroblasts.

4. The method according to claim 1, wherein the normal cells are salivary gland cells.

5. The method according to claim 3, wherein the salivary gland cells are those taken in vivo or those differentiated from stem cells.

6. The method according to claim 1, wherein the odontoma cells are those taken in vivo or those differentiated from stem cells.

7. A method for preparing salivary gland cells for use in transplantation, wherein a cell sample consisting of salivary gland cells is cultured in the presence of a Rho kinase inhibitor.

8. The method according to claim 7, wherein the Rho kinase inhibitor is (R)-(+)-trans-N-(4-pyridyl)-4-(1-aminoethyl)-cyclohexanecarboxamide or a salt thereof.

9. A material for regenerative medicine, which comprises salivary gland cells obtained by the method according to claim 7.

10. The method according to claim 1, wherein the culture is conducted to obtain cells for use in transplantation.

11. The material according to claim 9, which is to be transplanted into the Wharton's duct.
